SEQUENCE LISTING (2 sequences)

SEQ ID NO: 1 - protein, 6 aa, Artificial Sequence
  MOD_RES (1): ACETYLATION
DXLIXC

SEQ ID NO: 2 - protein, 6 aa, Artificial Sequence
  MOD_RES (1): ACETYLATION
DELIXC

(19) United States
(12) Patent Application Publication
(10) Pub. No.: US 2005/0004766 A1
(43) Pub. Date: Jan. 6, 2005

(54) Use of computationally derived protein structures of genetic polymorphisms in pharmacogenomics for drug design and clinical applications

(76) Inventors: Kalyanaraman Ramnarayan, San Diego, CA (US); Edward T. Maggio, San Diego, CA (US)

Correspondence Address: FISH & RICHARDSON, PC, 12390 EL CAMINO REAL, SAN DIEGO, CA, 92130-2081, US

(21) Appl. No.: 10/911,946

(22) Filed: Aug. 4, 2004

Related U.S. Application Data
(63) Continuation of application No. 09/704,362, filed on Nov. 1, 2000, now abandoned.
Application No. 09/704,362 is a continuation of application No. 09/438,566, filed on Nov. 10, 1999, now abandoned.

(51) Int. Cl. G06F 19/00; G01N 33/48; G01N 33/50

(52) U.S. Cl. 702/19; 702/20

(57) ABSTRACT

Provided herein are computer-based methods for generating and using three-dimensional (3-D) structural models of target biomolecules. In particular, the target biomolecules are protein structural variants derived from genes containing genetic variations, or polymorphisms. The models are generated using molecular modeling techniques, such as homology modeling. The models can be used in structure-based drug design studies to identify drugs that bind to particular structural variants in structure-based drug design studies, for designing allele-specific drugs, population-specific drugs and for predicting clinical responses in patients. Molecular structure databases containing protein structural variant models also are provided.

### RELATED APPLICATIONS

This application is a continuation of U.S. application Ser. No. 09/704,362 to Kalyanaraman Ramnarayan and Edward Maggio, filed Nov. 1, 2000, which is a continuation of U.S. application Ser. No. 09/438,566 to Kalyanaraman Ramnarayan, Edward Maggio and Patrick Hess, filed Nov. 10, 1999, entitled “USE OF COMPUTATIONALLY DERIVED PROTEIN STRUCTURES OF GENETIC POLYMORPHISMS IN PHARMACOGENOMICS FOR DRUG DESIGN AND CLINICAL APPLICATIONS.” The subject matter of each of the above-referenced applications is incorporated herein in its entirety.

### FIELD OF THE INVENTION

The present invention is related to computer-based methods and relational databases that use three-dimensional (3-D) protein structural models derived from genetic polymorphisms in the areas of computer-assisted drug design and the prediction of clinical responses in patients.

### BACKGROUND OF THE INVENTION

Recent advances in molecular biology, such as the discovery and identification of large numbers of genes and the sequences thereof encoded in the genomes of humans, other mammals and infectious disease agents, have contributed to the identification of a large number of proteins, biological receptors and other macromolecules and complexes that are promising therapeutic targets. Based on information derived from the gene sequences, the three-dimensional (3-D) molecular structures of the corresponding target proteins or receptors can be determined.

Since 3-D protein structure is related to biological function, structure-based drug design is an increasingly useful methodology that has made a great impact in the design of biologically active lead compounds. Drug designers can design and screen potential new drugs via computational methods, such as docking or binding studies, before actually beginning patient testing. These experiments can be performed in silico at a tiny fraction of the clinical cost.

The resulting molecules, while serving as lead compounds, often have unpredictable effects when employed in clinical trials. In addition, it has been observed that existing drugs with known clinical efficacy far often fail to achieve beneficial results when given to particular patients, or particular populations, such as ethnic groups, of patients. Genetic stratification of a population can be the difference between drug failure and drug approval.

The methods herein provide, among a variety of benefits, a means to address and solve these problems.

Accordingly, it is an object herein to provide methods for determining and utilizing protein 3-D structures that are derived from genetic polymorphisms to understand differences in biological activity that result from the polymorphisms, and to use this understanding to aid in the identification of potential new drug candidates and drug therapies.

It is thus a further object herein to provide methods for analyzing 3-D structures of protein structural variant targets derived from genetic polymorphisms to identify common structural features among the variants.

It is also an object herein to provide methods for identifying structural changes in target proteins that are associated with multiple mutations arising from genetic polymorphisms and correlating this information with biological activity.

It is an object herein to provide methods for using clinical data in conjunction with structural variants derived from genetic polymorphisms to understand and predict the pharmacological effects and clinical outcomes for drugs or potential drugs.

### SUMMARY OF THE INVENTION

Provided herein are computer-based methods for generating and using three-dimensional (3-D) structural models of target biomolecules. In particular, the target biomolecules are protein structural variants derived from genes containing genetic variations, or polymorphisms. The models are generated using molecular modeling techniques, such as homology modeling. The models are in structure-based drug design studies to design and identify drugs that bind to particular structural variants. They can also be used in structure-based drug design studies and to predict clinical responses in patients. They can also be used to design drugs that will bind to all or a substantial portion of allelic variants of a target, and hence to increase the population of patients for whom a particular drug will be effective and/or to decrease the undesirable side-effects in a larger population.

Hence, computer-based methods of drug design based on target protein structural models derived from genetic polymorphisms are provided. The methods involve obtaining one or more amino acid sequences of a target protein that is the product of a gene exhibiting genetic polymorphisms, where sequences represent different genetic polymorphisms, and generating 3-D protein structural variant models from the sequences. Structure-based drug design techniques are used to design potential new drug candidates or to suggest modifications to existing drugs based on predicted intermolecular interactions of the drugs or drug candidates with the models. Alternatively, drug molecules can be computationally docked with 3-D protein structural variant models from the sequences and energetically refined before performing structure-based drug design studies.

Genetic polymorphisms arise, for example, as a result of gene sequence differences or as a result of post-translational modifications, including glycosylation. Hence genetic polymorphisms are manifested as gene products and proteins having variant structures. The variant structures result in differences in biological responses among the originating organisms. These differences in response, include, but are not limited to, differences among patient responses to a particular drug, effective dosage differences, and side effects. With respect to infectious organisms, some polymorphisms may arise that convey resistance or susceptibility to particular drug therapies by altering the drug target structure.

Structural changes that arise as a result of genetic polymorphisms, are not of unlimited variety, since 3-D structure impacts upon function. A knowledge of the repertoire of the fine differences among generally similar 3-D structures of particular proteins will permit design of drugs that bind to the most polymorphisms, drugs that induce the fewest side-effects, and drugs that are more effective against infectious agents. Knowledge of these structures ultimately will permit patient-specific or subpopulation-specific, such as ethnic groups or age group, design or selection of drugs.

In preferred embodiments, binding interactions between a drug or potential new drug candidate molecules and the structural variants are calculated in order to optimize intermolecular interactions between drug or potential drug molecules and the structural variant models or to select drug therapies for patients by determining a drug or drugs that have favorable binding interactions with the structural variant models.

In other embodiments, the binding interactions are determined by calculating the free energy of binding between the protein structural variant model and a docked molecule; and decomposing the total free energy of binding based on the interacting residues in the protein active site.

After the protein structural variant models are generated, selected model structures can be analyzed to determine common structural features that are conserved throughout the selected models. The conserved structural features can serve as scaffolds or pharmacophore models into which potential drugs or modified drugs are docked. For example, the selected model structures may represent the structural variants resulting from the most commonly occurring genetic polymorphisms or from genetic polymorphisms found in a specific patient subpopulation. Alternatively, the models may be selected based on clinical information; for example, the structural variants may be derived based on patients receiving a specific treatment regimen or exhibiting a particular clinical response to a given drug, or on the duration of a particular drug treatment, or a particular age group or ethnic or racial group, sex or other subpopulation.

Also provided are relational databases for managing and using information relating to genetic polymorphisms. The databases contain 3-D molecular coordinates for structural variants derived from genetic polymorphism, a molecular graphics interface for 3-D molecular structure visualization, functionality for protein sequence and structural analysis and database searching tools. The databases may further include observed clinical data associated with the genetic polymorphism. The databases provide a means to design the allele-specific drugs and also to identify among alleles common or conserved structural features that can serve as the target for drug design.

The methods provided herein can be used for predicting clinical responses in patients based on genetic polymorphisms. For example, in preferred embodiments, a structural variant model derived from a patient exhibiting a particular genetic polymorphism is generated and screened against a number of reference protein structural variant models derived from genetic polymorphisms of the same gene in other patients. In certain embodiments, the reference structures are stored in a database along with observed clinical data associated with the structures, or polymorphisms. The patient structural variant model is compared to the reference structures, for example, by database searching, in order to identify reference structural variants that are similar to the model structure derived from the patient. Based on the premise that structurally similar targets will have similar clinical responses, a clinical outcome can be predicted for the patient based on the structures identified through structural comparison or database searching. This information can also be used in the design and analysis of clinical trials.

The methods can further be used to design therapeutic agents that are active against biological targets that have become drug resistant due to genetic mutations. In certain embodiments, 3-D protein structural variant models are generated for a target protein in which genetic mutations have occurred and against which a given drug is no longer biologically active. The models are compared to 3-D protein structural variant models of the target protein against which the drug has biological activity in order to identify structural differences between the susceptible and resistant targets. The differences can be used to understand the structural contributions to drug resistance, and this information can be utilized in structure-based drug design calculations to identify new drugs or modifications to the existing drug that circumvent the resistance problem.

A computer-based method for identifying compensatory mutations in a target protein is also provided. The method involves obtaining the amino acid sequence of a target protein containing multiple amino acid mutations that is expressed in a patient, where the structure of a form of the target protein that responds to a particular drug, including the active site, has been structurally characterized; generating a 3-D structural model of the mutated protein; comparing the structure of the mutated protein with the form of the protein that responds to the drug to identify structural differences and/or similarities arising from the mutations; comparing the biological activities of the drug against the mutated protein and the form of the protein that responds to the drug to determine the effects of the mutations on drug response; and identifying the mutations in the protein that affect biological activity based on the comparisons.

Molecular structure databases containing protein structural variant models produced by the methods also are provided. The databases may also contain biological or clinical data associated with the structural variants. The databases can be interfaced to a molecular graphics package for visualization and analysis of the 3-D molecular structural models. In particular, databases containing the 3-D structures of polymorphic variants of selected target genes, particularly pharmaceutically significant genes, such as proteases and polymerases, including reverse transcriptases, and receptors, such as cell surface receptors, are provided. The databases may be stored an provided on any suitable medium, including, but are not limited to, floppy disks, hard drives, CD-ROMS and DVDs.

Systems, including computers, containing the databases also are provided herein. Any computer known to those of skill in the art for maintaining such databases is contemplated.

### BRIEF DESCRIPTION OF THE DRAWINGS

FIG. 1 illustrates a method for creating a protein structural variant relational database.

FIG. 2 is a flow chart that describes one method used to generate structural variant models derived from genetic polymorphisms and to use the models in structure-based drug design studies.

FIG. 3 is a flow chart that describes an alternative method used to generate structural variant models derived from genetic polymorphisms and to use the models in structure-based drug design studies.

FIG. 4 shows the correlation between experimental and calculated changes of binding energy upon ligand modifications in the binding site of NS3.

FIG. 5 shows a comparison of calculated versus experimental binding free energy changes for complexes of the tumor necrosis factor (TNF) receptor with different inhibitors.

### DETAILED DESCRIPTION OF THE INVENTION

Definitions

Unless defined otherwise, all technical and scientific terms used herein have the same meaning as is commonly understood by one of skill in the art to which this invention belongs. All patents, patent applications, published patent applications and publications referred to herein are, unless noted otherwise, incorporated by reference in their entirety. In the event a definition in this section is not consistent with definitions elsewhere, the definition set forth in this section will control.

As used herein, polymorphism refers to a variation in the sequence of a gene in the genome amongst a population, such as allelic variations and other variations that arise or are observed. Genetic polymorphisms refers to the variant forms of gene sequences that can arise as a result of nucleotide base pair differences, alternative mRNA splicing or post-translational modifications, including, for example, glycosylation. Thus, a polymorphism refers to the occurrence of two or more genetically determined alternative sequences or alleles in a population. These differences can occur in coding and non-coding portions of the genome, and can be manifested or detected as differences in nucleic acid sequences, gene expression, including, for example transcription, processing, translation, transport, protein processing, trafficking, DNA synthesis, expressed proteins, other gene products or products of biochemical pathways or in post-translational modifications and any other differences manifested among members of a population. A single nucleotide polymorphism (SNP) refers to a polymorphism that arises as the result of a single base change, such as an insertion, deletion or change in a base.

A polymorphic marker or site is the locus at which divergence occurs. Such site may be as small as one base pair (an SNP). Polymorphic markers include, but are not limited to, restriction fragment length polymorphisms, variable number of tandem repeats (VNTR's), hypervariable regions, minisatellites, dinucleotide repeats, trinucleotide repeats, tetranucleotide repeats and other repeating patterns, simple sequence repeats and insertional elements, such as Alu. Polymorphic forms also are manifested as different mendelian alleles for a gene. Polymorphisms may be observed by differences in proteins, protein modifications, RNA expression modification, DNA and RNA methylation, regulatory factors that alter gene expression and DNA replication, and any other manifestation of alterations in genomic nucleic acid or organelle nucleic acids.

As used herein, structural variant proteins refer to a variety of 3-D molecular structures or models thereof that result from the polymorphisms. These variants typically arise from transcription and translation of genes containing genetic polymorphisms.

As used herein, binding interactions refer to atomic or physical interactions between molecules including, but not limited to, binding free energy, hydrophobic interactions, electrostatic interactions, steric interactions and other interactions that are commonly considered by those of skill in the art to determine the affinity of one molecule to bind to another. Favorable binding interactions refer to binding interactions that promote physical or chemical associations between molecules.

As used herein, a target protein is defined as a protein that is a receptor with which drugs or other ligands, such as small molecule or peptide agonists or antagonists or other proteins or biomacromolecules, such as DNA or RNA, interact to bring about a biological response.

As used herein, structure-based drug design refers to computer-based methods in which 3-D coordinates for molecular structures are used to identify potential drugs that can interact with a biological receptor. Examples of such methods include, but are not limited to, searching of small molecule libraries or databases, conformational searching of a ligand within an active site to identify biologically active conformations or computational docking methods.

As used herein, pharmacogenomics refers to the study of the variability of patient responses to drugs due to inherent genetic differences.

As used herein, computational docking refers to techniques wherein molecules, for example, a ligand and receptor or active site, are fitted together based on complementary interactions, for example, steric, hydrophobic or electrostatic interactions.

As used herein, energetic refinement refers to the use of molecular mechanics simulation techniques, such as energy minimization or molecular dynamics, or other techniques, such as quantum-based approaches, to “adjust” the coordinates of a molecular structural model to bring it into a stable, low energy, conformation. In molecular mechanics simulations, the potential energy of a molecular system is represented as a function of its atomic coordinates along with a set of atomic parameters, called a forcefield. Energy minimization refers to a method wherein the coordinates of a molecular conformation are adjusted according to a target function to result in a lower energy conformation. Molecular dynamics refers to methods for simulating molecular motion by inputting kinetic energy into the molecular system corresponding to a specified temperature, and integrating the classical equations of motion for the molecular system. During a molecular dynamics simulation, a system undergoes conformational changes so that different parts of its accessible phase space are explored.

As used herein, clinical data refers to information obtained from patients pertaining to pharmacological responses of the patient to a given drug, including, but not limited to efficacy data, side effects, resistance or susceptibility to drug therapy, pharmacokinetics or clinical trial results.

As used herein, compensatory mutations are mutations that act in concert with active site mutations by compensating for functional deficits caused by changes or mutations that affect binding in the active site.

As used herein, a relational database is a collection of data items organized as a set of formally-described tables from which data can be accessed or reassembled in many different ways without having to reorganize the database tables. Such databases are readily available commercially, for example, from Oracle, IBM, Microsoft, Sybase, Computer Associates or multiple other vendors.

A. Structure Generation and Analyses

As noted, patients exhibit variable responses to drugs. For some patients a drug may be very beneficial and achieve a desired response; whereas for other patients, with the same disorder, the same drug will have little or no effect. It is known that individuals as well as groups of individuals exhibit a variety of genetic polymorphisms. As described herein, the presence or absence of such polymorphism can be correlated with the variability of patient responses to drugs.

It is shown herein that by understanding how genetic polymorphisms affect 3-D protein structure of a drug target, for example, it is possible to ascertain the interaction of a particular drug with the target in a particular patient or groups of patients. Based upon this interaction, the outcome can be predicted. It will be possible to determine whether a patient will benefit from a drug or be at risk for a particular side effect. It is possible to predict these responses before exposure to the drug. These methods also permit rational design of drugs that can treat various populations or ultimately even individuals. These differences and effects can also be taken into account to design drugs that are not dependent upon a particular polymorphism.

Hence, the knowledge derived from understanding the effects of genetic polymorphisms can be used to develop and apply therapeutics more effectively, make clinical trials more successful, for example, by permitting selection of test subjects with the same polymorphism or with polymorphisms for which the drug is designed to interact effectively.

It is shown herein that it is advantageous to utilize 3-D molecular structures in drug design rather than to consider sequences alone. For example, most drugs target proteins, and disease, drug action and toxicity are all manifested at the protein level. Although the nucleotide sequences of genetic polymorphisms might appear to be quite different, the resulting protein targets may have similar shapes and, therefore, the protein biological function might be the same. Conversely, although genetic polymorphism sequences might appear similar, the resulting proteins may have critical differences in their 3-D structures that greatly affect biological activity.

Once the protein target structural models have been generated, structure-based drug discovery methodologies, for example, computational screening or docking, can then be used to design biologically-active compounds based on the 3-D structures of the biomolecular receptors. Using these methods, drug designers can identify and computationally rank various potential clinical drug candidates for maximum efficacy, thus cutting the time and expense associated with drug discovery.

In addition to drug design applications, the information derived from studying the structures of biological targets can be used to understand and predict biological responses in patients, such as efficacy, toxicity, drug resistance or other pharmacological effects. Since human clinical trials may cost upwards of $100-300 million, it is desirable to predict the outcome to the greatest extent possible for each prospective drug candidate so that the best prospective drug candidates are advanced to clinical trials.

1. Generating 3-D Protein Structural Variant Models

The first step in the methods provided herein is to obtain patient samples of a gene that exhibits genetic polymorphisms or of a therapeutic target protein derived therefrom. Starting with gene sequences that include single or multiple nucleotide polymorphisms, the amino acid sequences of the translated proteins can be determined. Alternatively, patient samples of the target protein can be obtained and sequenced directly. Multiple sequence analyses can be performed to determine the exact amino acid variations or mutations resulting from the genetic polymorphisms. Numerous methods for identifying genes that encode polymorphisms are known, numerous polymorphisms have been identified and mapped, and databases of such polymorphisms are publicly available.

3-D structural models of the native protein or of the protein structural variants are then determined, either through experimental methods, for example, x-ray crystallography or NMR, from a protein structure database, such as the PDB, or by using any of a number of well known techniques for predicting protein structure from sequence, for example, homology modeling, de novo protein folding algorithms and methodologies, and other computational protein structure prediction methods. Homology modeling techniques are among those preferred herein.

Homology Modeling

Homology modeling is based on the relationship between protein evolutionary origin, function and folding patterns. Proteins of related origin and function have conserved sequences and structural features among the members of a homologous family. Using these relationships, a three-dimensional structural model for a protein of unknown structure can be constructed by using composite parts of related proteins in the same family. Where only the primary amino acid sequence of a target protein is known, the sequence can be compared to the sequences of related proteins with known structures (reference proteins), and a model can be built by incorporating the structural attributes of the reference protein together with the sequence of the target protein.

Sequence homology calculations generally require: the amino acid sequence of the target protein; a high resolution structure for at least one, but preferably more, related reference proteins; and any other related amino acid sequences. The reference proteins include structures which are similar to the target protein, either by sequence, fold, function, or which are polymorphisms of the target protein. The more related protein structures and sequences that are available, the more reliable the technique will be at providing an accurate model.

In constructing a protein model using homology modeling, sequence alignment is performed between the target sequence and any known structures within the protein family. Sequence alignment requires determining the similarity between protein sequences by maximizing the number of matches between the sequences while introducing the minimum number of insertions and deletions. Sequence alignment algorithms are well known in the art, and standard gap penalties (i.e., programs automatically introduce gaps to maximize alignment and then adjust the percentage of identity by applying penalties for gap number and gap length) and other parameters can be selected by the skilled artisan. Additionally, the 3-D structures of the known reference proteins, preferably, are aligned to give the best overall fit for the proteins in the family. This provides indication of structurally-conserved regions, such as regions of the proteins that do not contain insertions or deletions, among the reference structures.

Once the sequences are aligned and the structurally-conserved regions are identified, the coordinates of the reference proteins can be used to construct a 3-D model of the target structure. Coordinates from the protein backbone of the reference proteins are then used to construct the backbone framework for the target protein structure. Side chains can be constructed, for example, by using side chain coordinates from the reference proteins, searching from a database to obtain side chain conformations that fit in with the existing structural framework or by generating side chains ab initio to establish energetically favorable side chain conformations.

The non-conserved regions of the unknown protein can be constructed, for example, using database searching. A database of known protein structures can be searched to identify variable regions in other proteins that have a high degree of sequence similarity to the target sequence and that fit onto the existing structural framework of the protein model. The variable regions can also be modeled by fitting the target sequence to a peptide backbone generated by varying phi and psi angles of the amino acids to give a loop structure that can be integrated into the model structure based on a sterically and energetically reasonable fit.

Algorithms for performing sequence similarity matching and homology model building are well known in the art and are available commercially (available from Molecular Simulations, Inc., Tripos, Inc. and from numerous academic sources).

Alternatively, ab initio methods can be used in combination with an existing partial homologous structure to generate unresolved portions of the target structure. Such methods are described, for example, in U.S. Pat. Nos. 5,331,573, 5,579,250 and 5,612,895, which as all patents, applications and publications referenced herein, are each incorporated in their entirety. These methods involve: simulating a real-size primary structure of a polypeptide in a solvent box, i.e., an aqueous environment; shrinking the size of the peptide isobarically and isothermally; and expanding the peptide to its real size in selected time periods, while measuring the energy state and coordinates, i.e., the bonds, angles and torsions of the expanding molecule. As the peptide expands to its full size, it assumes a stable tertiary structure. In most cases, due to the manner in which the expansion occurs, this tertiary structure will be either the most probable structure (i.e., it will represent a global minimum for the structure) or one of the most probable structures. The energy equations used to perform the ab initio simulation are based on the potential energy of the simulated molecule as described using molecular mechanics.

Once a model is built, it can be refined using energy minimization or molecular dynamics calculations. The steric and energetic quality of the structural models is then evaluated by analyzing the structural attributes of the model, such as phi and psi angles (e.g., by calculating Ramachandran or Balasubramanian plots), or the energetics of the model, such as by calculating energy per residue or strain energy. If the overall quality of the model is not satisfactory, further iterative energy refinement can be performed until the model is considered to be acceptable.

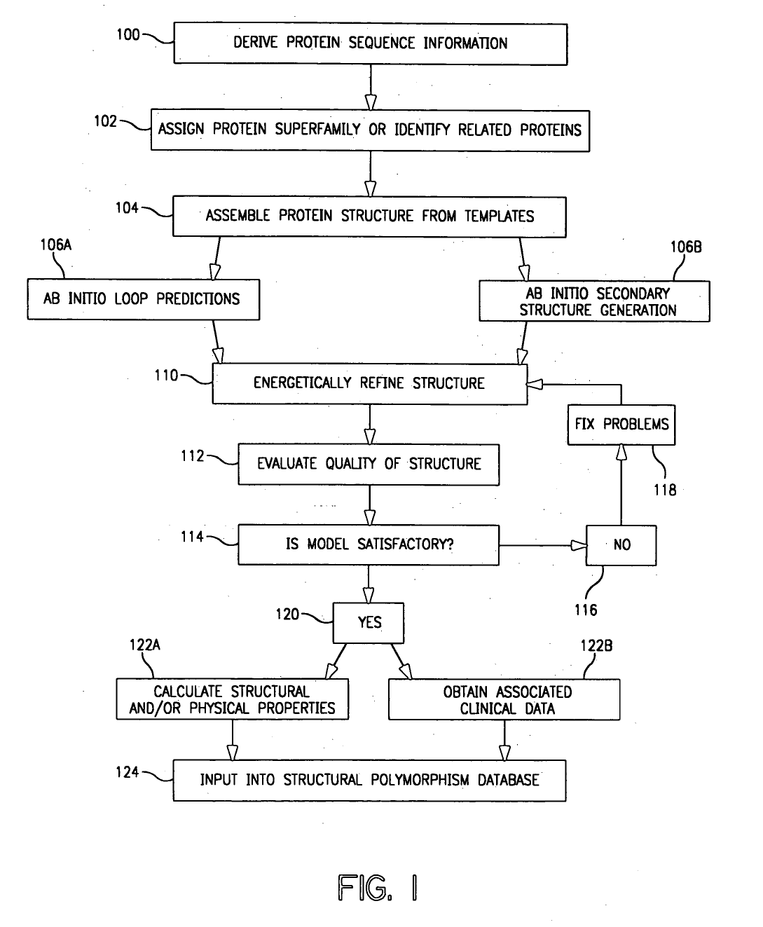

A preferred method for generating and refining the structural variant models is illustrated in FIG. 1. First, protein sequence information is derived based on the genetic polymorphisms. The subject protein is then assigned to a protein superfamily in order to identify related proteins to be used as templates to construct a 3-D model of the protein. If the superfamily is not known, sequence analysis or structural similarity searches can be performed to identify related proteins for use as templates in homology modeling studies. Once the conserved regions of the model are assembled, ab initio loop prediction or ab initio secondary structure generation techniques can be used to complete the model. Energetic refinement of the structure can be accomplished by performing molecular mechanics calculations, for example, using an ECEPP type forcefield or through molecular dynamics simulations, for example, using a modified AMBER type forcefield. If necessary, the structures can be dynamically refined, for example, by using a simulated annealing protocol (e.g., 100 ps equilibration, 500 ps dynamics, up to 1000° K., 1 fs data collection). For quality control, the protein structural characteristics, for example, stereochemistry e.g., phi/psi and side chain angles), energetics (e.g., strain energy), packing profile (e.g., packing factor per residue) and hydrophobic packing are evaluated and required to meet acceptable criteria before the structures are used in further studies or input into a structural polymorphism database.

2. Creating 3-D Structural Polymorphism Databases

After 3-D structural models are constructed for all protein structural variants, representing all known genetic polymorphisms, these can be inputted into a structural polymorphism relational database, along with associated structural or physical properties or clinical data (if available), as shown in FIG. 1. The databases can then be used to aid in structure-based drug design studies or for clinical analysis.

The database is preferably interfaced to a molecular graphics package that includes 3-D visualization and structural analysis tools, to analyze similarities and variations in the protein structural variant models. (see, copending U.S. application Ser. No. 09/272,814, filed Mar. 19, 1999, which is incorporated by reference herein in its entirety). Briefly, U.S. application Ser. No. 09/272,814 provides a database and interface for access to 3-D molecular structures and associated properties, which can be used to facilitate the design of potential new therapeutics. The interface also provides access to other structure-based drug discovery tools and to other databases, such as databases of chemical structures, including fine chemical or combinatorial libraries, for use in structure-focused high-throughput screening, as well as to a host of public domain databases and bioinformatics sites.

A relational database that collects multiple data files relating to the same molecular structure in the same subdirectory and that provides an interface to access all of the collected files from the same structure using the same user interface program is also provided. The collected files include a variety of information and computer file formats, depending on the type of information to be conveyed to users of the database. In practice, a user communicates over a public network, such as the Internet, or over a controlled network, such as an internet, with a secure file server that controls access to the collected files, and the interface to the collected files is provided by a standard graphical user interface program that is widely available. In this way, a convenient means of searching molecular structure data for characteristics of interest is provided. Data searching, file viewing, and investigation of multiple representations of molecular structures from within a single viewing program can also be performed using the database and interface.

The data files can be those available over a wide network such as the Internet, and the graphical user interface used for viewing the data files is a standard Internet web browser program, such as the web browser products by Netscape Communications, Inc. and Microsoft Corporation that are distributed free of charge. Such browser products readily import and provide views of files having a wide variety of formats that contain alphanumeric, video, and audio data. A security server is preferably located between the user browser program at a network client machine controls access to the database, which is housed at a file server connected to the security server. Before a user gains access to the database, the security server checks authorization for the individual user and then, if appropriate, permits downloading of appropriate data from the database file server.

Data for a molecular structure are loaded into the database by specifying the file pathnames for the various data files that contain the different types of data, including the different molecule views. Using a browser to view the data files permits various helper applications, called plug-ins, to smoothly and transparently accept the different file formats and provide views to the user. The various data files of the database are organized in accordance with the database design when they are loaded into the database and are managed by a relational database management program.

In addition to 3-D protein structures, as provided herein, the database can optionally contain associated biological or clinical data, such as drug resistance, side effects, efficacy, pharmacokinetics and other data, that correlate with or can be correlated with the structural variants. This information will be used for correlating observed clinical effects to specific structural variants and for predicting clinical responses and outcomes based on a patient's structural variants, i.e., genetic polymorphisms.

Structural analysis tools are preferably integrated with the structural database for comparing and analyzing the resulting protein structural variant models. For example, the molecular graphics software package described in copending U.S. application Ser. No. 09/272,814, includes structural analysis capability to measure the structural attributes of the model (distances, angles, etc.), to analyze sequences and secondary structures, to study physical properties such as hydrophobicity, electrostatic potential, and active or reactive sites in the protein, as well as to evaluate the quality of the structure (both conformationally and energetically).

Structures can also be compared by aligning them, such as by performing a least squares fitting of the x-, y- and z-coordinates of each of the structural variant models and superimposing the structures or any other alignment method or structural comparison method. For example, the structures of the variants can be clustered, or grouped together, based on structural similarity. This can save time over studying each structural variant independently because, where structures are considered to be similar enough that they are clustered together (e.g. if their structures can be superimposed within a specified tolerance), then only a representative structure, or perhaps an average structure or scaffold, which is derived as a composite of the individual structural variant models, can be used in further drug design studies.

Tools for database searching can also be included in the software package. These can be used to query the database for structural variant models having similar properties, such as molecular structure or sequence similarity. These tools are used, for example, to mine the database to identify variant models that are structurally similar (e.g. to find structures that overlap within a specified tolerance), and thus would be predicted to interact in the same way with potential drugs or exhibit the same clinical response. This information could be useful in understanding the structural or clinical effects of different genetic polymorphisms and could potentially save time and money by extending the results of previously performed clinical or computer-based drug design studies to predict the results of studies on similar structural variants that have not yet been performed.

3. Selecting Relevant Structural Variants

The structural variant models can be used to design new drugs or to select a drug therapy that would be appropriate for a patient exhibiting a particular genetic polymorphism. As it may not be possible for a drug to work equally well for all polymorphisms, and thus all patients, representative structural variants can be selected for use in drug design studies in order to maximize biological activity based on genetic polymorphisms.

In some cases, the structural variant corresponding to the genetic polymorphism occurring most commonly in a population can be used to identify drugs that would be effective in the greatest percentage of the population. Optionally, structures corresponding to a relevant subpopulation, such as a particular gender, age, race, or other characteristic, can be selected for use in designing drugs that are active in that subpopulation. In other cases, individual structural variant models can be used to design drugs that are specifically active against one target in one individual arising from a particular genetic polymorphism.

The relevant structural variants may be identified using the structural analysis tools described herein, optionally in combination with database and statistical analysis tools that permit a complete analysis and comparison of the molecular structures and properties of the structural variants.

B. Use of Structural Variant Models in Structure-Based Drug Design

The structural differences in protein structural variants that arise due to genetic polymorphisms can have profound effects on biological activity. Because of the structural differences among the variants, they may have different physical or reactive properties and therefore may exhibit different biological activities. These differences may include, for example, different responses to a given drug, so that a drug which works well in a patient with one particular genetic polymorphism may not work as well in another patient exhibiting a different polymorphism.

The 3-D molecular structures of drug targets derived from genetic polymorphisms can be used in structure-based drug design studies to greatly advance the development of new pharmaceuticals. Relational databases of these 3-D structures that are derived from samplings of genetic polymorphisms over a patient population or a cross-section of the population can be used to design potential drugs in order to optimize effectiveness for the particular population.

The structures and databases described herein can provide information that is useful, for example, in designing a drug that is effective in the greatest percentage of the population. It is desirable that a given drug is effective in the largest percentage of the population, since such a drug is likely to have the greatest clinical utility and thus the greatest commercial value. A drug with superior performance properties is sometimes referred to as a “best in class” drug and is highly prized by pharmaceutical companies since this heralds market leadership and the likelihood of commercial success. The databases and methods described herein can be used to determine 3-D protein structures for drug targets that are associated with particular genetic polymorphisms and to use the structures in drug design studies for design and optimization of candidate drugs that exhibit activity over the broadest patient population.

Genetic polymorphisms may result in target protein structural variants in which drug efficacy correlates with specific populations or subpopulations. In some cases, it might be desirable to target drug design or drug therapy toward a specific patient population affected by a certain disease or condition, such as a particular race or gender, or toward those having a specific genetic polymorphism. The information derived from comparing the 3-D structural variants arising from different genetic polymorphisms may be useful for understanding why drugs are active or inactive in different subpopulations, or for assisting in developing new drugs to maximize efficacy across specific populations.

It is also possible to individualize drug design or drug therapy by determining the structural variants associated with a particular patient and then designing or screening drugs or potential drugs to maximize efficacy in that subject or in a subpopulation that exhibits the same genetic polymorphism.

The variants may also be used to track polymorphic variations in infectious organisms, such as viruses. For example, the human immunodeficiency viruses (HIVs) reverse transcriptase and protease have served as drug targets (see, Erickson et al. (1996) Ann. Rev. Pharmacol. Toxicol 36:545-571); their three-dimensional structures are known (see, e.g., Nanni et al. (1993) Perspectives in Drug Discovery and Design 1:129-150; Kroeger et al. (1997) Protein Eng. 10:1 379-1383). The clinical emergence of drug-resistant variants of these viruses has limited the long-term effectiveness of drugs targeted against these enzymes.

As noted, these enzymatic proteins in order to preserve function must exhibit conserved 3-D structures. The methods herein permit design of drugs specific for the conserved regions of the 3-D structures. They also permit selection of drug regimens based upon the alleles expressed. Hence, methods for designing HIV enzyme-specific drugs are provided.

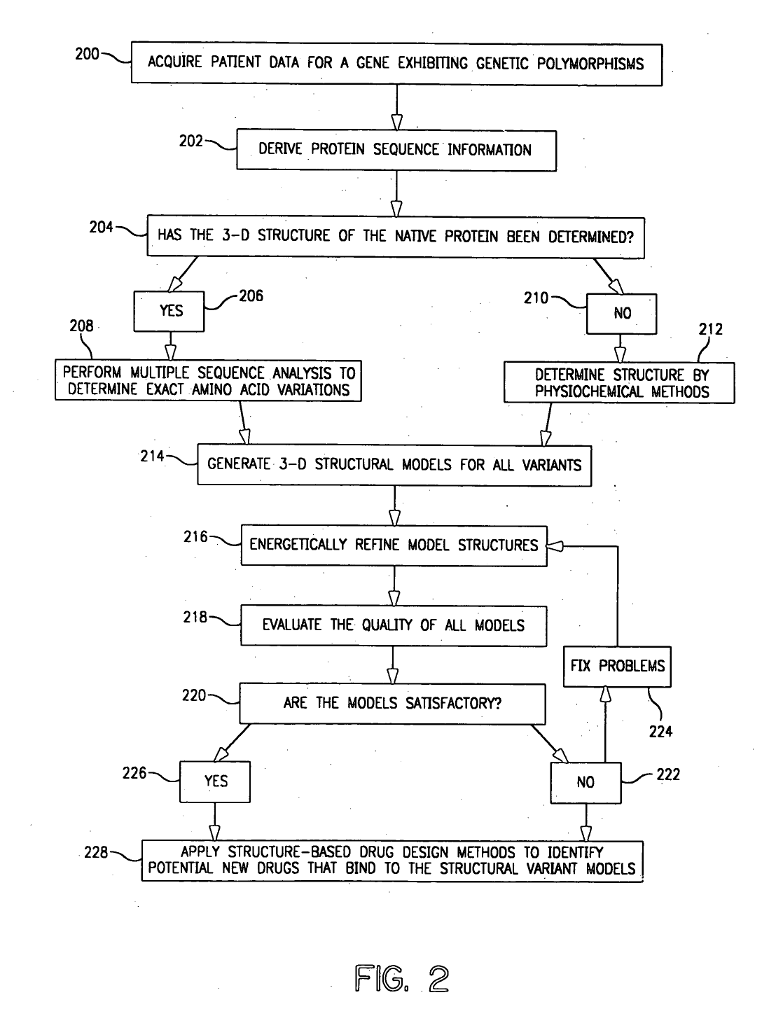

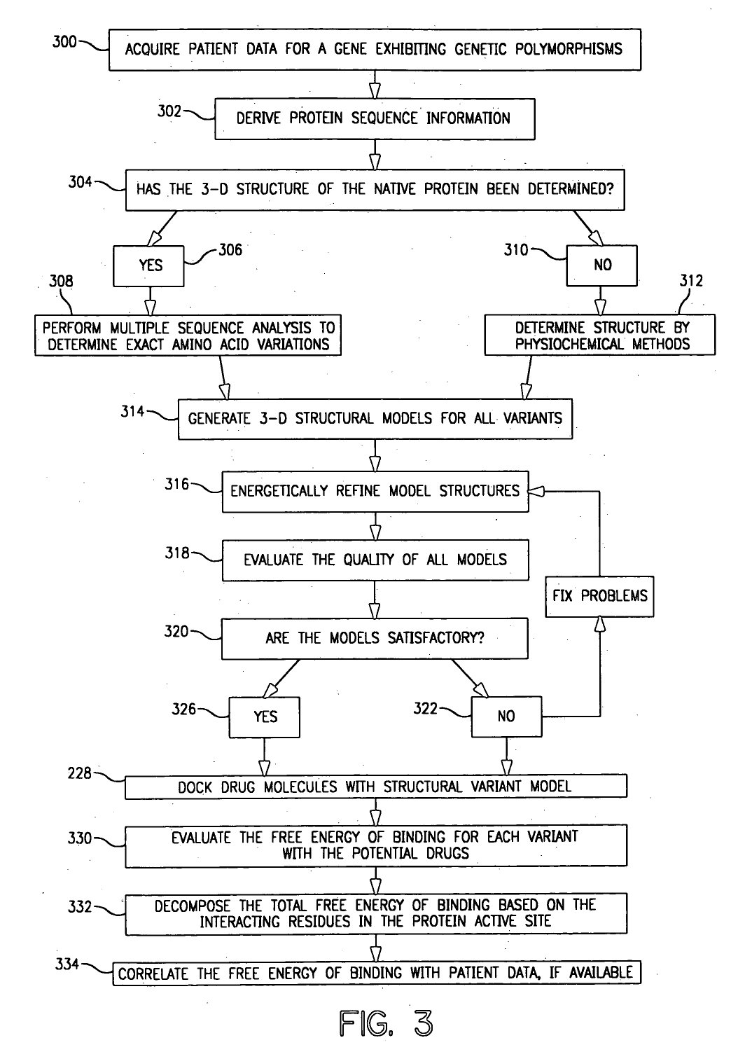

Flow charts illustrating alternative embodiments for using protein 3-D structures derived from genetic polymorphisms in structure-based drug design studies are given in FIGS. 2 and 3.

1. Computational Docking and Binding Studies

The structural variant models can then be used to design potential new drugs or to aid in the selection of a drug therapy based on the interactions of selected small molecules with the particular variants.

Structure-based drug design experiments, such as computational screening or docking studies, calculation of binding energies or analysis of steric, electrostatic or hydrophobic properties of the resulting structural variant models, can be performed on selected structural variant models to aid in the understanding of observed biological activities or to determine new potential drug candidates to bind to the particular target. Methods for performing such studies are well known and software tools for performing the calculations are widely available.

For example, new potential drug candidates can be designed by identifying potential small molecule drugs that can bind to a particular structural variant. This is accomplished, for example, through electronic screening of small molecule databases or other methods known to those of skill in the art to determine molecules that would have optimal binding interactions with the structural variant target.

In certain preferred embodiments, the free energy of binding of different drugs or potential drugs to each structural variant model can be calculated. The total free energy of binding is decomposed based on the interacting residues in the protein active site (see, e.g., Wang et al. (1996) J. Am. Chem. Soc. 118:995-1001; Wang et al. (1995) J. Mol. Biol. 253:473-492; Ortiz et al. (1995) J. Med. Chem. 38:2681-2691, which describes a computational method for deducing QSARs from ligand-macromolecule complexes).

2. Identifying Conserved Structural Features or Pharmacophores

In comparing sets of related protein structures, such as those with the same biological function or those resulting from genetic polymorphisms, certain parts of the structural framework are often found to be conserved, while other parts vary among the proteins. Mutations that occur in the conserved regions of the structure can have significant effects biological activity. For example, in viruses, the conserved features can be essential to protein function, and thus to the viability of the infectious organism or virus. Identifying the conserved structural features over a range of structures often gives insight into which structural features are necessary for biological activity, and are therefore non-mutatable. By analyzing a number of structural variants derived from genetic polymorphisms that exhibit drug resistance, it is possible to identify or design drugs that interact best with the common structural features in all of the variants. Using these features in structure-based drug design studies leads to the identification of drugs that retain biological activity despite multiple mutations, or polymorphisms, and could help to overcome the problem of drug resistance.

In certain preferred embodiments, new potential drug candidates can be identified using the structural variant models by identifying pharmacophores or conserved features in the protein structural variant models and using this structural information to identify small molecules that would bind to the structural variant models.

Using structural comparison tools described above, the common structural features that are conserved across a range of structural variant models of a given protein based on different genetic polymorphisms can be identified. To do this, multiple structural variant models are compared, generally by superimposing the coordinates of one variant model onto those of one or more other variants and observing the structural fit. Such functionality is commonly found in molecular graphics or homology modeling packages. Once the optimum fit of structures is performed, then the structural features that are present throughout the structural variant models can be identified and used as the basis for drug interactions in structure-based drug design studies. For example, the pharmacophores or conserved features can be specified as database queries and a library or database of small molecule structures can be searched to identify new lead compounds to bind to the pharmacophores. Alternatively, other structure-based ligand design strategies can be employed to design lead compounds or to identify modifications to be made to existing drugs to improve biological activity.

Following the computational drug design studies, any potential new drugs that are identified can be synthesized and subjected to further biological testing, such as in vitro studies or pre-clinical and clinical in vivo testing.

3. Applications in Drug Resistance

Where drug resistance that arises due to mutations or polymorphisms is observed, the methods described herein can be used to develop new drugs that overcome the resistance. For example, once drug resistance is observed, the structure associated with the resistant polymorphism can be determined and used in further drug design studies to suggest new drugs or modifications to the existing drug that will restore biological activity by targeting different mutants or that will target multiple mutants simultaneously.

The model structures can also be used to correlate drug resistance in infectious diseases with the structural variants derived from genetic polymorphisms. Here, the 3-D structure of the virus or other drug target is determined for the particular variant model against which the drug was effective. When drug resistance arises due to a genetic polymorphism, a model for the structure variant associated with the resistant organism can be generated, and a new drug can be designed or modifications can be made to the existing drug to overcome the resistance.

For example, samples of the mutating organism can be obtained over time and structural models for the resulting proteins can be generated. These models can then be used to design new drug therapies that are active against the mutated organism. Multiple drug resistant structures can be analyzed to obtain an average structure or to identify common structural features in order to design new drugs that have the broadest spectrum of activity against multiple mutations.

Such structural information is useful in designing effective drug therapies to overcome resistance or to develop drugs that are effective over a range of genetic polymorphisms and thus work for the maximum number of patients.

4. Identifying Compensatory Structural Changes

The methods described herein can be used to study the effects of multiple genetic polymorphisms on a resultant protein structure. Multiple mutations are common in AIDS and other viruses, which makes sequence correlation difficult. By observing the structural effects of the mutations on the resulting protein, it is possible to look at the net effect of all structural changes and to consider the overall structure of the protein in drug design studies. For example, a mutation might occur in the active site, or site of drug action, in a protein. Additionally, there may be related mutations in other parts of the protein structure, which might not be identified from a single point mutation correlation. These related mutations could have an effect on biological activity of the protein. By looking only at the active site, it might be predicted that a drug or potential drug would not bind to the protein. The additional mutation, however, might cause compensatory structural changes in the protein structure that alter its properties in a way that restores biological activity.

By computing 3-D protein structures from gene sequences containing multiple polymorphisms, it is possible to more accurately predict the effect of multiple sequence mutations on protein structure and, thus, to obtain a better correlation between sequence and drug resistance than by considering sequence correlations alone. This information can be useful, for example, in understanding drug resistance and can aid researchers and clinicians in developing new drug therapies to overcome drug resistance.

The structures that are derived based on multiple generic polymorphisms can be used in structure-based drug design studies to provide frameworks, or scaffolds, into which drug or potential drug molecules can be docked. This permits the design of drugs that are active against a wider range of structural variants, thus, in more patients or against a range of drug resistant proteins.

C. Applications of the Methods Genetic Polymorphisms and Structure-Based Drug Design

As noted above, structure-based drug design is an increasingly useful methodology that has made a great impact in the design of biologically active lead compounds. Drug designers can design and screen potential new drugs via computational methods, such as docking or binding studies, before actually beginning patient testing.

The drugs designed by such methods, and also those identified by traditional methods of drug discovery, are then tested in clinical trials. Among those that show efficacy for a particular indication and low toxicity ultimately are approved for use. It is found, however, that not all patients with a particular indication respond uniformly to the drugs. The drug may not be efficacious or side-effects may be pronounced.

The methods provided herein, represent a further advance in the use of rational drug design methods. As described herein, polymorphic variation has an effect upon the 3-D structure of encoded proteins. As a result, drugs interact with variants differently, leading to differential responses in the population as a whole. A new approach to drug design and testing is provided herein by identifying polymorphisms, and determining 3-D resulting structures, which are then used in computation drug design or in selection of patient populations or in designing treatment protocols or other applications.

Drug Resistance

In the case of infectious organisms or other replicating or mutating agents, such as flu, HIV, rhinovirus or biological warfare agents, some polymorphisms or mutations may arise over time which convey resistance or susceptibility to specific drug therapy, for example, by altering the drug target structure or physical properties so that a specific drug or therapy, such as an antibiotic or vaccine, may no longer be able to bind to or otherwise interact with the target protein to exert its desired biological effect. For certain infectious diseases, such as AIDS, genetic polymorphisms give rise to drug resistance as the virus mutates.

It is a further object herein to provide methods for understanding and overcoming drug resistances by using 3-D protein model structures resulting from multiple genetic polymorphisms or mutations in an infectious organism, and using this information in drug design studies.

Conserved Structural Features

If common structural features are observed over a range of protein targets that are derived from genetic polymorphisms, these common features may be used to design a drug that is effective with a variety of genetic polymorphisms and thus many patients. The retention of certain common structural features over a large number of genetic polymorphisms suggests that those features may not be mutatable because the conserved structure may be essential to protein function, e.g., to the viability of an infectious organism or virus. Such conserved structural elements are prime targets for structure-based drug design, e.g., anti-infective or antibiotic drug design, and can lead to highly effective therapies.

The common structural features can serve as a basis for structure-based drug design, for example, by serving as a scaffold, for building a receptor model into which potential drug candidates can be docked, or as a pharmacophore query for screening a library of physical or virtual chemical or biochemical molecules to identify compounds that match the pharmacophore template and, thus, are potential drug candidates.

Analysis of 3-D protein structural variants derived from genetic polymorphisms to identify the common structural features over a large number of structural variants can aid in the design of drugs that are active over a broad range of genetic polymorphisms, such as in a large number of patients or against drug resistant targets.

Effects of Multiple Polymorphisms on Protein Structure and Biological Activity

Certain proteins, for example, viral proteins or other infectious organisms, may harbor multiple genetic polymorphisms. Since each genetic polymorphism can give rise to slight changes in structure, some, and over time, many, additional genetic polymorphisms may cause changes in the protein structures that significantly affect biological activity. These structural changes could result in, for example, different dynamical behavior, alteration in enzyme kinetics or differences in substrate recognition, which can significantly alter drug response. For example, a mutation for one drug compound can suppress a mutation to a second drug due to compensatory effects. In these cases, a drug which is predicted to be ineffective for a given patient based upon the single nucleotide correlation may, in fact, be effective as a result of these changes.

Because mutations are so frequent in AIDS and other viruses, few sequences are exactly the same in different patients. Thus, it is difficult or inconclusive to generate multiple mutation sequence correlations for drug resistance. If each patient has a different viral sequence due to a high viral mutation rate, then no sequence correlation is even possible in such cases.

Clinical Applications A knowledge of the repertoire of structural differences arising from genetic polymorphisms across the human population or specific subpopulations can provide insight into the differing biological responses in patients based on their genetic differences. For example, where clinical data are available for patients having particular genetic polymorphisms, this information can be associated with the 3-D protein structural variants and used to find correlations between polymorphisms and observed drug responses.

The methods provided herein can be used to design drug therapies that bring about favorable clinical responses (or eliminate unfavorable effects) in patients, to identify pharmacological effects of drugs in different patient subpopulations and to simulate clinical trails to increase the probability that the trials will yield optimal results.

Due to the high cost of clinical trials, such studies are generally focused on small patient populations. The structural analysis tools described herein permit the extension of clinical trials to cover patient populations not specifically included in the study. This is accomplished through correlation of the structural variants derived from genetic polymorphisms with clinical responses.

The molecular structures and databases described herein can also find application in the understanding and prediction of clinical or pharmacological drug responses, for example, efficacy, toxicity, dose dependencies or side effects in patients. For example, relational databases containing 3-D protein structural variants can provide a means for managing and using the information to understand and predict clinical responses in patients.

In other embodiments, observed clinical data from patients in a clinical trial can be associated with the structural variant models for each genetic polymorphism exhibited in the clinical subjects, for example, in a structural polymorphism relational database. The correlation between the structural variants and observed clinical effects can then be utilized to predict clinical outcomes in patients that did not participate in the clinical trial. For example, a structural variant model can be generated for a patient based on a genetic polymorphism exhibited in the patient, and the database can be mined to identify structurally similar variants for which clinical results are known. Structural similarity can be determined, for example, by superimposing the structures and measuring the RMS differences between the structures or by using pattern matching or motif searching algorithms. The results can then be used to predict clinical responses in the patient based on the clinical data associated with the structurally similar variants.

The predicted correlations can also be used to aid in the design of subsequent clinical trials. The follow-up trials can be made more effective through the judicious selection of patients with given genotypes (i.e., those exhibiting the same genetic polymorphisms), as guided by the structurally predicted outcomes. For example, a clinical trial can be designed based on a subpopulation of clinical subjects which exhibit a specific genetic polymorphism (i.e. structural variant) to demonstrate the effectiveness of a given therapeutic on a targeted population.

In other embodiments, the methods provided herein can be used in the selection of drug therapies for patients exhibiting a particular genetic polymorphism. This is accomplished by generating the structural variant model associated with the polymorphism, docking drug molecules that might be used to treat the patient into the structural variant model and calculating the binding energies of each drug with the variant. The results of docking or free energy calculations can be correlated to clinical data, for example, patient population (e.g., ethnic background, race, sex, age), treatment regimen, patient response to a particular drug or duration of treatment. The binding energies can be compared, for example, to determine which drug would best bind to the variant in order to identify the drug that could best be used to treat the patient to optimize biological activity.

Computer Systems and Database

Databases containing data representative of the 3-D structure of structural variants encoded by a selected gene or genes or the 3-D structure of other polymorphic variants are provided. The selected genes can be drug target, such as receptors and genes of infectious agents, such as the HIV protease or reverse transcriptase. The databases may be stored on any suitable medium and used in any suitable computer system. Systems and methods for generating, storing and processing databases are well known.

The processing that maintains the database and performs the methods and procedures using the databases may be performed on multiple computers, or may be performed by a single, integrated computer. For example, the computer through which data is added to the database may be separate from the computer through which the database is sorted or analyzed, or may be integrated with it. Each computer operates under control of a central processor unit (CPU), such as a “Pentium” microprocessor and associated integrated circuit chips, available from Intel Corporation of Santa Clara, Calif., USA. A computer user can input commands and data from a keyboard and display mouse, and can view inputs and computer output at a display. The display is typically a video monitor or flat panel display device. The computer also includes a direct access storage device (DASD), such as a fixed hard disk drive. The memory typically includes volatile semiconductor random access memory (RAM). Each computer preferably includes a program product reader that accepts a program product storage device from which the program product reader can read data (and to which it can optionally write data). The program product reader can inclulde, for example, a disk drive, and the program product storage device can comprise removable storage media such as a magnetic floppy disk, an optical CD-ROM disc, a CD-R disc, a CD-RW disc, or a DVD data disc. If desired, computers can be connected so they can communicate with each other, and with other connected computers, over a network. Each computer can communicate with the other connected computers over the network through a network interface that permits communication over a connection between the network and the computer.

The computer operates under control of programming steps that are temporarily stored in the memory in accordance with conventional computer construction. When the programming steps are executed by the CPU, the pertinent system components perform their respective functions. Thus, the programming steps implement the functionality of the system as described above. The programming steps can be received from the DASD, through the program product reader, or through the network connection. The storage drive can receive a program product, read programming steps recorded thereon, and transfer the programming steps into the memory for execution by the CPU. As noted above, the program product storage device can include any one of multiple removable media having recorded computer-readable instructions, including magnetic floppy disks and CD-ROM storage discs. Other suitable program product storage devices can include magnetic tape and semiconductor memory chips. In this way, the processing steps necessary for operation can be embodied on a program product.

Alternatively, the program steps can be received into the operating memory over the network. In the network method, the computer receives data including program steps into the memory through the network interface after network communication has been established over the network connection by well known methods that will be understood by those skilled in the art without further explanation. The program steps are then executed by the CPU to implement the processing of the Garment Database system.

The following examples are included for illustrative purposes only and are not intended to limit the scope of the invention.

### EXAMPLE 1

Binding Correlations of Mutant Forms of HCV Protease with Different Inhibitors

Introduction

During HCV replication, the final steps of processing are performed by a virally encoded chymotrypsin-like serine protease NS3. NS3 is an approximately 3000 amino acid protein that contains, from the amino terminus to the carboxy terminus, a nucleocapsid protein (C), envelope proteins (E1 and E2) and several non-structural proteins (NS1, 2, 3, 4a, 4b, 5a and 5b). NS3 is an approximately 68 kda protein, encoded by approximately 1893 nucleotides of the HCV genome, and has two distinct domains: (a) a serine protease domain containing approximately 200 of the N-terminal amino acids; and (b) an RNA-dependent ATPase domain at the C-terminus of the protein. The NS3 protease is considered a member of the chymotrypsin family and is a serine protease that is responsible for proteolysis of the polypeptide (polyprotein) at the NS3/NS4a, NS4a/NS4b, NS4b/NS5a and NS5a/NS5b junctions responsible for generating four viral proteins during viral replication. This protease is inhibited by N-terminal cleavage products of substrate peptides. The NS3 protease, which is necessary for polypeptide processing and viral replication has been identified, cloned and expressed (see, e.g., U.S. Pat. No. 5,712,145).

Active NS3 forms a heterodimer with a polypeptide cofactor NS4A. The crystal structure of NS3 with and without the NS4A cofactor is known (see, e.g., Love et al. (1996) Cell 87:331-342; Habuka et al. (1997) Jikken Igaku 15:2308-2313; Yan et al. (1998) Protein Sci. 7:837-847, which provides the structure with NS4A).

The NS3 protease is a target for design of antiviral drugs. For example, a series of potent hexapeptide inhibitors of NS3 has been developed by optimization of the product inhibitors (Ingallinella et al. (1998) Biochemistry 37:8906-8914).

This example provides the results of a theoretical study of NS3 protease complexes with two peptide inhibitors described by Ingallinella et al. ((1998) Biochemistry 37:8906-8914). Models of the complexes were obtained by flexible docking of the peptides into the active site of the crystal structure of NS3/4A, followed by evaluation of protein-peptide binding energies. The models were tested by in situ modification of the docked ligands. A qualitative agreement between the binding energies and inhibitor IC₅₀ values obtained from literature was found.

The peptides studied were:

| Sequence* | IC⁵⁰, nM | SEQ ID |
|---|---|---|
| Ac-Asp¹-D-Glu²-Leu³-Ile⁴-Cha⁵-Cys⁶-COO- | 15 | 1 |
| Ac-Asp¹-L-Glu²-Leu³-Ile⁴-Cha⁵-Cys⁶-COO- | 60 | 2 |

*Cha = β-cyclohexylalanine

In the modeling studies, it was assumed that:
the high-affinity inhibitory peptides 1 and 2 have a similar mode of binding to the active site of NS3;
the minimum binding pharmacophore includes the SH group of Cys⁶and carboxyl groups of Asp¹, Glu² and Cys⁶; and
the side chains of residues 3, 4 and 5 may enhance binding by non-specific hydrophobic interaction with NS3.
   Methods

Initial Structure of the NS3-Peptide Complex

The crystal structure of NS3/NS4A was regularized using molecular mechanics. Peptides were placed into the NS3 binding site by analogy with other serine proteases:
the C-terminal carboxyl was placed near the oxyanion-stabilizing site (residues 137-139);
the side chain of Cys⁶ was inserted into the hydrophobic cavity formed by L135, F154 and A157; and
the ε-amino group of K136 was placed in contact with the C-terminal carboxyl (see, Steinkuhler et al. (1998) Biochemistry 37:8899)

Monte Carlo Simulations

Monte Carlo (MC) simulations were performed on the NS3-peptide complexes. The forcefield used was ECEPP/3 with modifications. The sampling method was biased probability Monte Carlo with random change of one variable at a time. A Metropolis acceptance criterion was applied after energy minimization (quasi-Newton, up to 1000 steps). Simulations were performed at a temperature of 1000° K. In the peptide, translation/rotation and all torsions were included in the simulation. Protein side-chain X angles of residues that have at least one atom within 7.0 Å of any atom of the peptide were included. The energy function used in the MC simulations included:
ECEPP/3 terms for energy in vacuo (VDW, H-bond, electrostatic and torsion potentials);
distance dependent electrostatics with e=4.0; and
surface energy with atomic solvation parameters.

The total energies of the complexes were calculated including contributions from: ECEPP/3 VDW, H-bond, S—S bond and torsion terms; exact-boundary electrostatic energy with e=8.0; and side-chain entropies. Hydrophobic free energies were estimated as sA, where A is accessible surface area and s is a tension constant of 0.03 kcal/molÅ².

Strategy of the Flexible Monte Carlo Docking

The simulations proceeded with multiple, relatively short MC runs (2000-5000 generated structures). New docking cycles were started from the lowest-energy or other interesting structures found in previous runs. Structures saved during various MC runs were sorted by total energies and RMSD, and compressed into a cumulative conformational stack. Binding energies were calculated for representative structures of each complex thus obtained. This strategy was more efficient than continuous long simulations because the variable torsion angles and distance constraints are defined for an initial structure and do not change during the MC run.

Binding Energies of the Peptide-Protein Complexes

Binding energies were estimated using the equation:
E_{bind}=Eₒ+E_{compl}−Eₚₑₚₜ−Eₚᵣₒₜ,
where E_{compl} is the energy of the complex, Eₚₑₚₜ & Eₚᵣₒₜ are separate energies of the peptide and protein, respectively, and Eₒ is an adjustable constant.

The binding energy function included: exact-boundary electrostatic contributions; side-chain entropy; and surface tension hydrophobic terms. ECEPP/3 hydrogen-bonding terms were included with a weight of 0.5.

Results

Models of the NS3-Peptide Complexes

RMSD between pharmacophore atoms of peptides 1 and 2 were calculated for all pairs of MC structures. The pairs of structures were selected with RMSD≦2.0 Å for the minimum set of pharmacophore atoms and with binding energies ΔE_{bind}<5.0 kcal/mol. Two models of the NS3-peptide complex were selected by visual inspection.

Characteristics of the binding sites for peptide inhibitors in two NS3-peptide complex models are summarized in Table 2.

TABLE 2

| | | | | Present for | |
|---|---|---|---|---|---|
| | Peptide | | Type of | Peptide | |
| site | residue | NS3 residue, group | interaction | Model 1 | Model 2 |
| P1 | Cys⁶COO⁻ | K136 NH₃+ | H-bond/el. | 1, 2 | 1, 2 |
| | | G137 NH | H-bond | 1, 2 | 2 |
| | | S139 OH | H-bond | 1, 2 | 2 |
| | Cys⁶SH | L135, F154, A157 | hydroph | 1, 2 | 1, 2 |
| P2 | Cha⁵ | H57, R155, A156 | hydroph | 1, 2 | — |
| | | A157, V158 | hydroph | — | 2 |
| P3 | Ile⁴ | V132, S133 | hydroph | 1, 2 | 2 |
| | | V158, C159 | hydroph | — | 1 |
| P4 | Leu³ | Res. 157 to 160 | hydroph | 1, 2 | 2 |
| | | V132, S133 | hydroph | — | 1 |
| P5 | Glu²COO- | R161 guanidine | H-bond/el. | — | 1, 2 |
| P6 | Asp¹COO- | R161 guanidine | H-bond/el. | 1, 2 | — |
| | | S133 OH | H-bond | — | 1, 2 |

Validation of the Models: Modifications of the Protein and Ligands in the Binding Site

Mutation K136M and peptide modifications known from structure activity relationship (SAR) studies were performed in low-energy structures of the NS3-peptide 2 complex.

Positions of the modified ligand and conformations of adjacent protein side chains were adjusted by energy minimization. Distance restraints were applied to keep the ligand near its initial position.

Changes in calculated binding energies upon modifications, ΔE_{bind} (calc), were compared to the values expected from ratios of inhibitory potencies, ΔE_{bind}(exp).
ΔE_{bind}(exp)=RT In(IC₅₀^{mod}/IC₅₀^{o}),
where IC₅₀^{o} and IC₅₀^{mod} are inhibitory potencies of the parent and modified compounds.

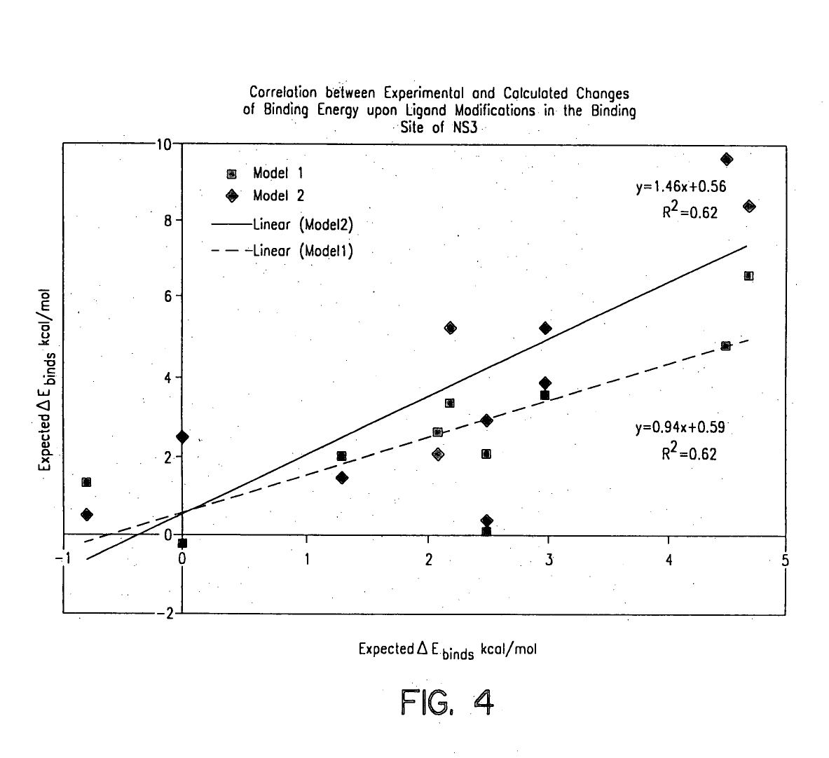

The correlation between experimental and calculated changes in binding energy upon ligand modifications in the binding site of NS3 is illustrated in FIG. 4.

Discussion

The two NS3-peptide complex models suggest a common binding pattern for the inhibitor P1 site (Cys⁶-OH) with the carboxyl group hydrogen-bonded to the oxyanion hole residues G137 and S139, and the Cys⁶ side chain embedded in a hydrophobic pocket formed by L135, F154 and A157.

This study confirms the possibility of hydrogen bonding between the C-terminal carboxyl and ε-amino group of K136 suggested by Steinkuhler et al. ((1998) Biochemistry 37:8899)) based on the K136M mutation in NS3. Changes in calculated binding energies upon mutation are consistent with an 8-fold increase in K₁ of an inhibitor with a free carboxyl group, and with the lack of an effect on binding when the peptide is amidated.

The models differ in binding of the negatively charged side chains in positions P5 and P6. The R161 guanidine interacts with a carboxyl group of Asp¹ and Glu² in Models 1 and 2, respectively. In Model 2, the Asp¹ carboxyl also interacts with the hydroxyl of S133.

The models are in agreement with SAR data for peptide inhibitors of NS3. Predicted changes in binding energy upon modification of the protein and peptides correlate reasonably well with the changes expected from IC⁵⁰ ratios. Standard deviations of ΔE_{bind}(calc)−ΔE_{bind}(exp) were 0.8 and 1.6 kcal/mol for Models 1 and 2, respectively, with correlation coefficients of 0.62. After the largest outlier was removed from each dataset, correlations improved to 0.81 and 0.76, respectively.

Conclusions

An effective iterative Monte Carlo protocol for the docking of flexible peptide ligands into a flexible protein active site has been developed. Two models of the complexes of HCV NS3 protease with potent peptide inhibitors were proposed based on the docking simulations and on evaluation of protein-ligand binding energies. The models were validated by in situ modifications of NS3-peptide complexes and by correlation of binding energies of modified complexes with those expected from experimental IC₅₀ values. Proposed models can be used for planning further mutagenesis studies of the HCV NS3 protease and the models can be used in the design of non-peptide inhibitors using structure-based drug design methodologies.

### EXAMPLE 2

Lead Optimization by Receptor-Based Free Energy Quantitative Structure Activity Relationships (QSARS) for Tumor Necrosis Factor (TNF) Receptor Antagonist Finding

The goal of the modeling studies in this phase was to discover the binding modes and complex structures of the compounds that bind to TNF receptor type I protein, in order to guide design of new compounds. An approach that relies on docking compounds to the receptor, evaluating free energy changes of binding of the docked structures, and comparing the calculated values with experimental inhibition constants Kᵢ of the compounds was developed. The success of the calculations was evaluated by the consistency of the calculated free energy changes of binding and the experimental Kᵢ.

The difference in free energy changes of binding between two compounds with inhibition constants Kᵢ and Kᵢ′ can be calculated as,
ΔΔ G=−kT InKᵢ′/Kᵢ
where k and T are Boltzmann's constant and absolute temperature, respectively.

The 13 active compounds were studied. Their potencies, as measured by Kᵢ, range from 0.1 to 30 μM, spanning about 3 kcal/mol in free energy. It was found that the calculated free energy changes of binding are highly consistent with the corresponding experimental values, with correlation coefficient 0.966 and difference less than 0.5 kcal/mol (see Table 1 and FIG. 4). The predicted binding modes and complex structures can thus be accepted with confidence.

To modify these compounds, important pharmacophore features on the surface of the receptor that are critical for binding of the compounds were identified. These features include a hydrophobic belt, a hydrophilic belt and 3 hydrogen bond donor sites. A few of potential hydrogen bonding sites, which are not used by the current compounds, were also derived, and can be used for designing more potent binders.

Graphics-guided redesign of the compounds was performed. The free energy calculation was used to predict the binding activity of each design. Fourteen new compounds were thus designed and binding activities were predicted. The chemical structures of the designed molecules, together with the binding modes of the lead compounds, were synthesized and shown to have high affinity for the target. Some of them exhibit a Kᵢ in low-nanomolar range. Hence the method provided herein for modification of drugs for binding to calculated 3-D structures of a target protein resulted in redesigned drug candidates with enhanced affinity for the target.

This approach has advantages over the traditional x-ray crystallography method, which include the following:
(1) The binding modes are determined for a group of compounds instead of single compound; analysis of similarity and differences reveals rich information in binding mechanisms.
(2) The predictive power of the free energy calculation is very desirable for redesign of compounds.
(3) The correlation with the biochemical activities assures relevancy of the explored binding modes, while a structure given by x-ray crystallography may not necessarily be one related to the biological functions of the compound.

A comparison of calculated relative free energy changes of binding ΔΔA and experimental ΔΔG converted from inhibition constants Kᵢ (all in kcal/mol) of the compounds (referenced by a code name) is presented in Table 3.

TABLE 3

| Compound | ΔΔA | ΔΔG |
|---|---|---|
| SBI-2030 | 0 | 0 |
| SBI-2002 | −0.97 | −1.25 |
| SBI-2005 | −0.72 | −1.14 |
| SBI-307 | −0.56 | −0.08 |
| SBI-2008 | −0.53 | −0.82 |
| SBI-2006 | −0.34 | −0.44 |
| SBI-306 | −0.07 | 0.40 |
| SBI-2000 | 0.29 | 0.27 |
| SBI-2001 | 0.72 | 1.12 |
| SBI-304 | 1.55 | 1.45 |
| SBI-308 | 1.70 | 1.78 |
| SBI-305 | 1.86 | 1.67 |
| SBI-2048 | 1.95 | 1.94 |

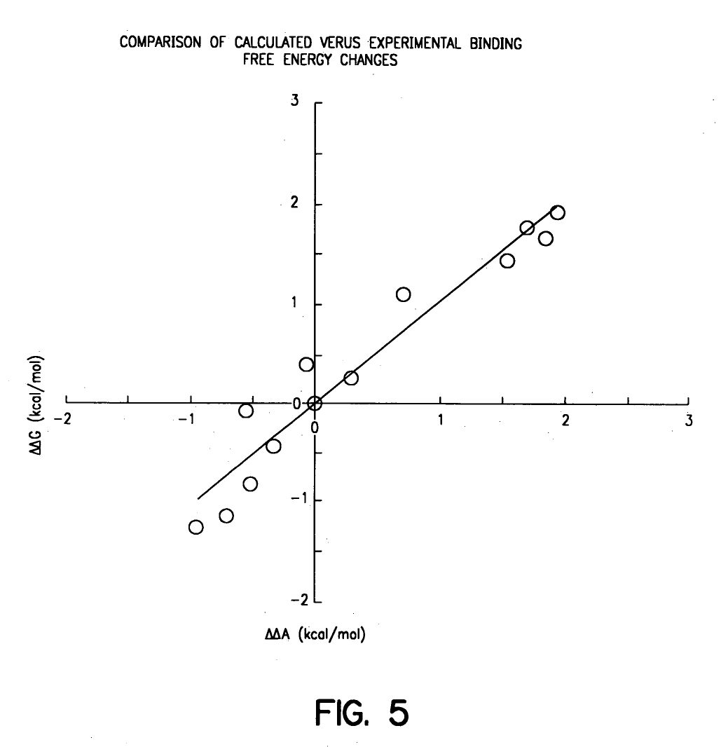

A comparison of calculated versus experimental binding free energy changes is given in FIG. 5.

Since modifications will be apparent to those of skill in this art, it is intended that this invention be limited only by the scope of the appended claims.

1. A computer-based method for predicting clinical responses in patients based on genetic polymorphisms, comprising:
identifying proteins that are the products of a gene exhibiting genetic polymorphisms;
obtaining amino acid sequences of the proteins that are the products of a gene exhibiting genetic polymorphisms;
determining 3-dimensional (3-D) protein structural variant models for the proteins that are the products of a gene exhibiting genetic polymorphisms;
building a relational database of protein structural variants based on genetic polymorphisms and observed clinical data associated with particular polymorphisms exhibited in the patients, wherein the database comprises:
3-D molecular coordinates for structural variant-drug complex models; and
observed clinical data associated with the genetic polymorphisms;
obtaining a protein structural variant encoded by a gene exhibiting genetic polymorphisms in a patient;
determining a 3-D protein model based on the patient's gene sequence of a protein exhibiting polymorphisms;
screening or comparing the 3-D model of the protein from the patient to the structures contained in the database by:
identifying structures in the database that are similar to the model derived from the patient's protein; and
predicting a clinical outcome for the patient based on the clinical data associated with the identified structures.

2. The method of claim 2, further comprising:
providing the database with a molecular graphics interface that interfaces with the database for 3-D molecular structure visualization;
providing the database with functionality that interfaces with the database for protein sequence and structural analysis; and
providing the database with searching tools that interface with the database.

3. The method of claim 1, wherein the step of determining 3-D protein structural variant models is performed by a method selected from the group consisting of experimental methods, searching protein structure databases, homology modeling, molecular modeling, de novo protein folding, computational protein structure prediction, ab initio methods and combinations thereof.

4. The method of claim 3, wherein the experimental methods include x-ray crystallography and NMR spectroscopy.

5. The method of claim 1, wherein the step of determining 3-D protein structural variant models is performed by a combination of homology modeling and ab initio methods.

6. The method of claim 1, wherein the database further comprises 3-D molecular structural data of structural variant models.

7. The method of claim 2, wherein the step of determining 3-D protein structural variant models is performed by a method selected from the group consisting of experimental methods, searching protein structure databases, homology modeling, molecular modeling, de novo protein folding, computational protein structure prediction, ab initio methods and combinations thereof.

8. The method of claim 7, wherein the experimental methods include x-ray crystallography and NMR spectroscopy.

9. The method of claim 2, wherein the step of determining 3-D protein structural variant models is performed by a combination of homology modeling and ab initio methods.

10. The method of claim 2, wherein the database further comprises 3-D molecular structural data of structural variant models.

11. A computer-based method for predicting clinical responses in patients based on genetic polymorphisms, comprising:
determining a 3-D protein model based on a patient's gene sequence of a gene that exhibits polymorphisms;
screening or comparing the 3-D model of the protein from the patient to the 3-D structures contained in a database of 3-D protein structures by:
identifying structures in the database that are similar to the model of the protein derived from the patient; and
predicting a clinical outcome for the patient based on clinical data associated with the identified structures.

12. The method of claim 11, wherein the step of determining a 3-D protein model is performed by a method selected from the group consisting of experimental methods, searching protein structure databases, homology modeling, molecular modeling, de novo protein folding, computational protein structure prediction, ab initio methods and combinations thereof.

13. The method of claim 12, wherein the experimental methods include x-ray crystallography and NMR spectroscopy.

14. The method of claim 11, wherein the step of determining a 3-D protein model is performed by a combination of homology modeling and ab initio methods.
